(19) United States
(12) Patent Application Publication
(10) Pub. No.: US 2010/0004485 A1
(43) Pub. Date: Jan. 7, 2010

(54) GABAPENTIN ENACARBIL SALTS AND PROCESSES FOR THEIR PREPARATION

(75) Inventors: Meital COHEN, Petach-Tikva (IL); Valerie NIDDAM-HILDESHEIM, Kadima (IL); Maytal PIRAN, Rishon Le Zion (IL); Elena BEN MOHA-LERMAN, Kiryat Ono (IL)

Correspondence Address: MERCHANT & GOULD PC, P.O. BOX 2903, MINNEAPOLIS, MN, 55402-0903, US

(73) Assignee: TEVA PHARMACEUTICAL INDUSTRIES LTD., Petach-Tikva (IL)

(21) Appl. No.: 12/496,883

(22) Filed: Jul. 2, 2009

Related U.S. Application Data
(60) Provisional application No. 61/133,948, filed on Jul. 2, 2008.
(60) Provisional application No. 61/134,255, filed on Jul. 7, 2008.
(60) Provisional application No. 61/134,354, filed on Jul. 8, 2008.
(60) Provisional application No. 61/190,966, filed on Sep. 3, 2008.
(60) Provisional application No. 61/196,433, filed on Oct. 16, 2008.

(51) Int. Cl. C07C 229/28

(52) U.S. Cl. 562/507

(57) ABSTRACT

The preparation and use of calcium, barium, magnesium and copper salts of gabapentin enacarbil are described.

### CROSS-REFERENCE TO RELATED APPLICATIONS

This application claims the benefit of U.S. Provisional Patent Application Ser. Nos. 61/133,948, filed Jul. 2, 2008; 61/134,255, filed Jul. 7, 2008; 61/134,354, filed Jul. 8, 2008; 61/190,966, filed Sep. 3, 2008; and 61/196,433, filed Oct. 16, 2008, all of which are incorporated herein by reference in their entirety.

### FIELD OF THE INVENTION

The present invention relates to gabapentin enacarbil salts, their preparation and their use in the preparation of gabapentin enacarbil.

### BACKGROUND OF THE INVENTION

Gabapentin (GBP), 1-(aminomethyl)cyclohexaneacetic acid is described according to the following formula:

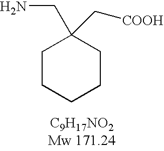

GBP is a white to off-white crystalline solid with a pKa1 of 3.7 and a pKa2 of 10.7. GBP is marketed by Pfizer under the trade name Neurontin®.

GBP is used in the treatment of cerebral diseases such as epilepsy. In animal models of analgesia, GBP prevents allodynia (pain-related behavior in response to a normally innocuous stimulus) and hyperalgesia (exaggerated response to painful stimuli). GBP also decreases pain related responses after peripheral inflammation. Animal test systems designed to detect anticonvulsant activity proved that GBP prevents seizures as do other marketed anticonvulsants.

Gabapentin enacarbil (GBPE), 1-{[(α-isobutanoyloxyethoxy)carbonyl]-aminomethyl}-1-cyclohexane acetic acid, is a Transported Prodrug of GBP and is described according to the following formula:

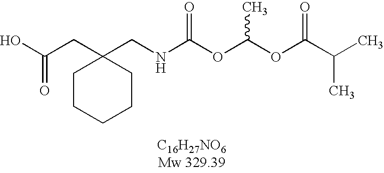

GBPE is designed to improve some of the bioavailability limitations that are known in GBP. GBPE is designed to be recognized by high-capacity transport proteins expressed all along the intestinal tract, making it suitable for sustained-release formulation for colonic absorption. After absorption GBPE is rapidly converted to GBP.

GBPE and processes for its preparation are described in U.S. Pat. No. 6,818,787 (parallel to PCT publication no. 2002/100347 “WO '347”); U.S. Pat. Nos. 7,232,924 and 7,227,028. U.S. Published Application No. US 2005/0154057 describe a crystalline form of GBPE.

U.S. Pat. No. 6,818,787 also describes pharmaceutically acceptable salts of GBPE, specifically GBPE-Na, hydrates and solvates of GBPE and process for the preparation of GBPE-Na using water and 0.5N NaHCO₃.

The occurrence of different crystal forms (polymorphism) is a property of some molecules and molecular complexes. A single molecule may give rise to a variety of solids having distinct physical properties like melting point, X-ray diffraction pattern, infrared absorption fingerprint and NMR spectrum. The differences in the physical properties of polymorphs result from the orientation and intermolecular interactions of adjacent molecules (complexes) in the bulk solid.

Accordingly, polymorphs are distinct solids sharing the same molecular formula yet having distinct advantageous and/or disadvantageous physical properties compared to other forms in the polymorph family. One of the most important physical properties of pharmaceutical polymorphs is their solubility in aqueous solution, which may influence the bioavailability of the drug.

These practical physical characteristics are influenced by the conformation and orientation of molecules in the unit cell, which defines a particular polymorphic form of a substance. The polymorphic form may give rise to thermal behavior different from that of the amorphous material or another polymorphic form. Thermal behavior is measured in the laboratory by such techniques as capillary melting point, thermogravimetric analysis (TGA) and differential scanning calorimetric (DSC) and can be used to distinguish some polymorphic forms from others. A particular polymorphic form may also give rise to distinct spectroscopic properties that may be detectable by powder X-ray crystallography, solid state ¹³C NMR spectrometry and infrared spectrometry.

The discovery of new polymorphic forms of pharmaceutically acceptable salts of GBPE provides a new opportunity to improve the performance of the active pharmaceutical ingredient (API), by producing polymorphs of pharmaceutically acceptable salts of GBPE having improved characteristics, such as flowability, and solubility. Thus, there is a need in the art for polymorphic forms of pharmaceutically acceptable salts of GBPE.

There is a need in the art for additional salts of GBPE, as well as using them in crystallization processes.

### SUMMARY OF THE INVENTION

In one embodiment, the present invention encompasses GBPE salt selected from the group consisting of: GBPE-Ca salt, GBPE-Ba salt, GBPE-Mg salt and GBPE-Cu salt.

In another embodiment, the present invention encompasses a solid salt of GBPE selected from the group consisting of: GBPE-Ca, GBPE-Ba, GBPE-Mg and GBPE-Cu.

In yet another embodiment, the present invention encompasses amorphous GBPE salt selected from the group consisting of: GBPE-Ca, GBPE-Ba, GBPE-Mg and GBPE-Cu.

In one embodiment, the present invention encompasses isolated GBPE salt selected from the group consisting of: GBPE-Ca, GBPE-Ba, GBPE-Mg and GBPE-Cu.

In another embodiment, the present invention encompasses pure GBPE salt selected from the group consisting of: GBPE-Ca, GBPE-Ba, GBPE-Mg and GBPE-Cu.

In yet another embodiment, the present invention encompasses a process for preparing GBPE salt selected from the group consisting of GBPE-Ca, GBPE-Ba, GBPE-Mg and GBPE-Cu comprising: dissolving GBPE in an organic solvent; adding a base selected from the group consisting of NaOH, KOH, K₂CO₃, KHCO₃, Na₂CO₃, NaHCO₃ and mixtures thereof; adding water and adding any one of the following salts: CaCl₂, BaCl₂, MgCl₂ and Cu(OAc)₂.

In one embodiment, the present invention provides a process for preparing GBPE salt selected from the group consisting of GBPE-Ca, GBPE-Ba and GBPE-Mg comprising: dissolving GBPE in a water miscible organic solvent or mixtures of a water miscible organic solvent and water; adding a base selected from the group consisting of NaOH, KOH, K₂CO₃, KHCO₃, Na₂CO₃, NaHCO₃, LiOH, Li₂CO₃ and mixtures thereof; and adding CaCl₂ if the GBPE salt is GBPE-Ca, BaCl₂ if the GBPE salt is GBPE-Ba, or MgCl₂ if the GBPE salt is GBPE-Mg.

In another embodiment, the present invention encompasses a process for preparing GBPE-Mg salt or GBPE-Cu salt comprising: dissolving GBPE in a water immiscible organic solvent; adding a base selected from the group consisting of NaOH, KOH, K₂CO₃, KHCO₃, Na₂CO₃, NaHCO₃, LiOH, Li₂CO₃, Cu(OAc)₂, Mg(OEt)₂ and mixtures thereof; and adding Cu(OAc)₂ if the GBPE salt is GBPE-Cu or Mg(OEt)₂ if the GBPE salt is GBPE-Mg.

In yet another embodiment, the present invention further provides a process for preparing GBPE-Mg salt or GBPE-Cu salt comprising: dissolving GBPE in a water immiscible organic solvent; and adding Cu(OAc)₂ if the GBPE salt is GBPE-Cu or Mg(OEt)₂ if the GBPE salt is GBPE-Mg.

In one embodiment, the present invention encompasses a process for crystallizing GBPE by seeding it with a salt selected from the group consisting of GBPE-Ca, GBPE-Ba, GBPE-Mg and GBPE-Cu. Preferably, the process comprises combining the GBPE with a linear, branched or cyclic C₅ to C₁₂ alkane to obtain a reaction mixture; and seeding the reaction mixture with a GBPE salt selected from the group consisting of GBPE-Ca, GBPE-Ba, GBPE-Mg and GBPE-Cu.

In another embodiment, the present invention encompasses a pharmaceutical composition comprising GBPE salt selected from a group consisting of: GBPE-Ca salt, GBPE-Ba salt, GBPE-Mg salt and GBPE-Cu salt.

In yet another embodiment, the present invention encompasses the use of a GBPE salt selected from a group consisting of: GBPE-Ca salt, GBPE-Ba salt, GBPE-Mg salt and GBPE-Cu salt in the manufacture of a pharmaceutical composition.

### BRIEF DESCRIPTION OF THE FIGURES

FIG. 1 depicts characteristic X-ray powder diffraction pattern of gabapentin enacarbil (“GBPE”) Ca salt amorphous form.

FIG. 2 depicts characteristic X-ray powder diffraction pattern of GBPE-Ba salt amorphous form.

FIG. 3 depicts characteristic X-ray powder diffraction pattern of GBPE-Mg salt amorphous form.

FIG. 4 depicts characteristic X-ray powder diffraction pattern of GBPE-Cu salt amorphous form.

FIG. 5 depicts thermogravimentric analysis (“TGA”) of amorphous GBPE.

FIG. 6 depicts thermogravimetric analysis of GBPE Ba salt.

### DETAILED DESCRIPTION OF THE INVENTION

As used herein, the term “GBPE” refers to 1-{[(α-isobutanoyloxyethoxy)carbonyl]-aminomethyl}-1-cyclohexane acetic acid.

As used herein, the term “pure” refers to a product having a purity of above about 98%, preferably, above about 99.5%, more preferably, above about 99% as measured by HPLC.

As used herein, the term “room temperature” refers to a temperature of about 15° C. to about 30° C., preferably, about 20° C. to about 25° C.

As used herein, the term “over night” refers to a period of about 10 hours to about 20 hours, preferably to a period of about 14 hours to about 16 hours.

As used herein, the term “sparingly soluble” refers to a compound having a solubility of about 0.01 g/ml to about 0.03 g/ml.

As used herein, the term “freely soluble” refers to a compound having a solubility of about 0.1 to about 1 g/ml.

As used herein the term “isolated” in reference to GBPE salt refers to GBPE salt that is physically separated from the reaction mixture where it is formed. For example, the separation can be done by filtering the precipitated GBPE salt.

The present invention encompasses GBPE salt selected from the group consisting of: GBPE-Ca salt, GBPE-Ba salt, GBPE-Mg salt and GBPE-Cu salt.

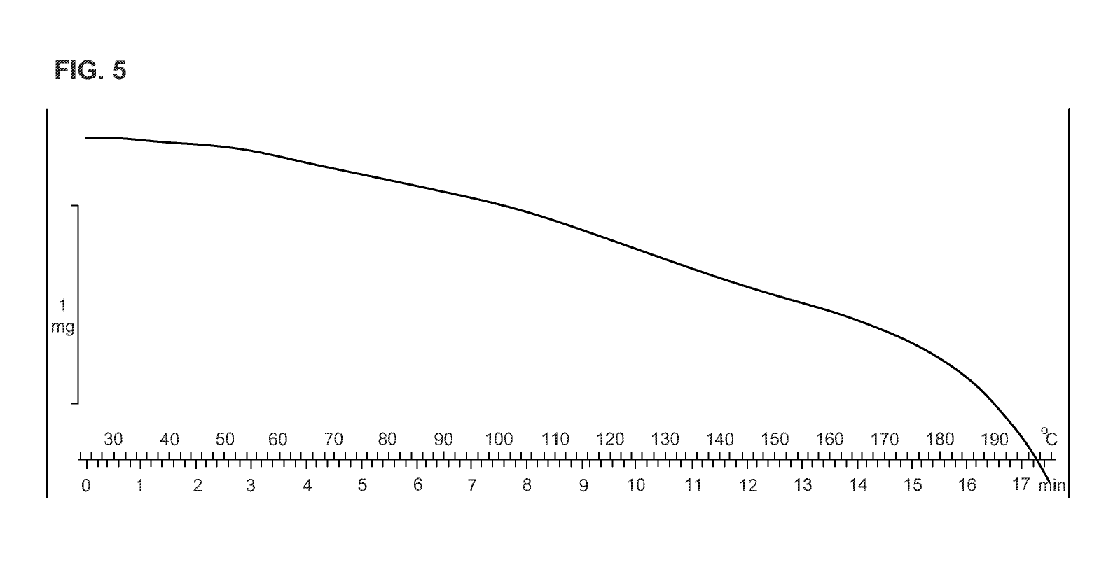

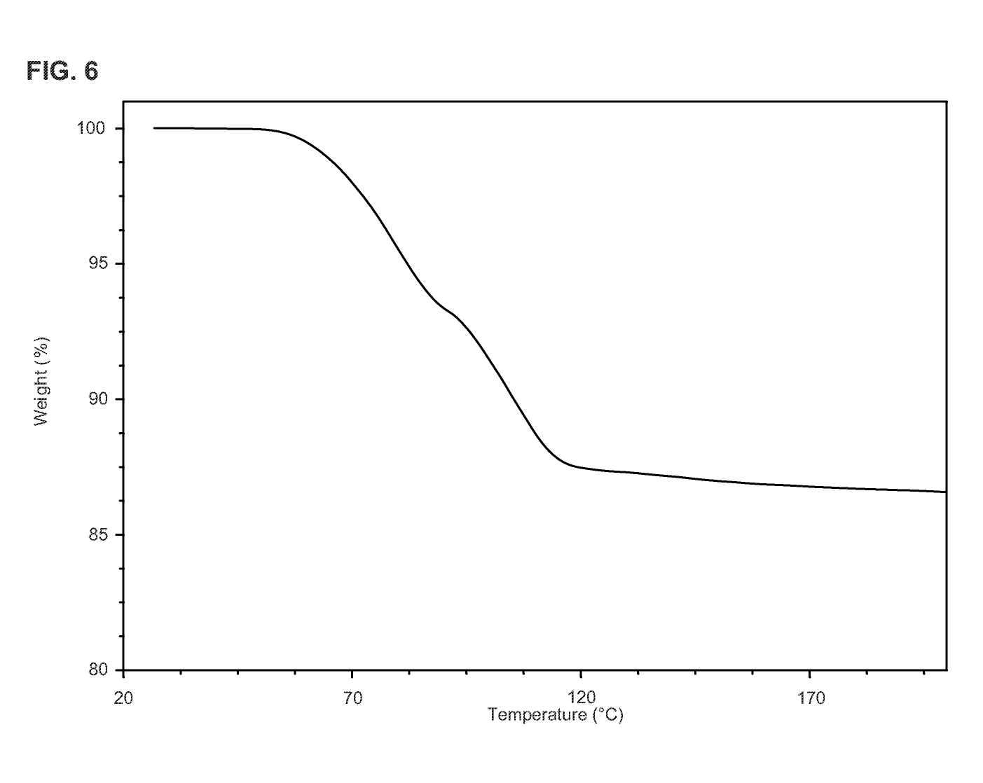

GBPE salts disclosed in the present invention possess distinct advantageous elements, such as increased stability and solubility in water, compared to GBPE amorphous. The presently claimed GBPE salts demonstrate increased thermal stability, as well as physical and chemical stability when compared to GBPE amorphous. The presently claimed salts start to decompose at much higher temperatures than that of GBPE amorphous, which shows decomposition from a temperature of about 30° C. and above, as can be seen in FIGS. 5 and 6. In addition, GBPE amorphous (described in the WO '347 patent) is in an oil form, while the salts of the present invention are in solid form.

During water solubility tests, GBPE salts of the present invention were found to be more soluble than GBPE amorphous itself. GBPE Ca, Cu and Mg salts were found to be sparingly soluble in water, GBPE Ba salt is freely soluble in water, while GBPE amorphous was found to be insoluble in water, as presented in Table 1. This solubility makes the presently claimed salts of GBPE better seeding agents, as well as easier to handle in an industrial pharmaceutical process, than the GBPE amorphous of the prior art.

GBPE-Ca salt is less hygroscopic when compared to GBPE sodium salt known in the art, thus making it more suitable for seeding. GBPE sodium salt is difficult to handle during production because of its extremely hydrophilic nature. In addition, GBPE-Ca salt is less soluble than the sodium salt, also contributing to its effectiveness as a seeding agent. GBPE-Ba salt shows stability over a long period of time, retaining its form during a long period of storing. GBPE-Ba-salt was also found to be non-hygroscopic, and contained no water when kept in room conditions, as shown in FIG. 6.

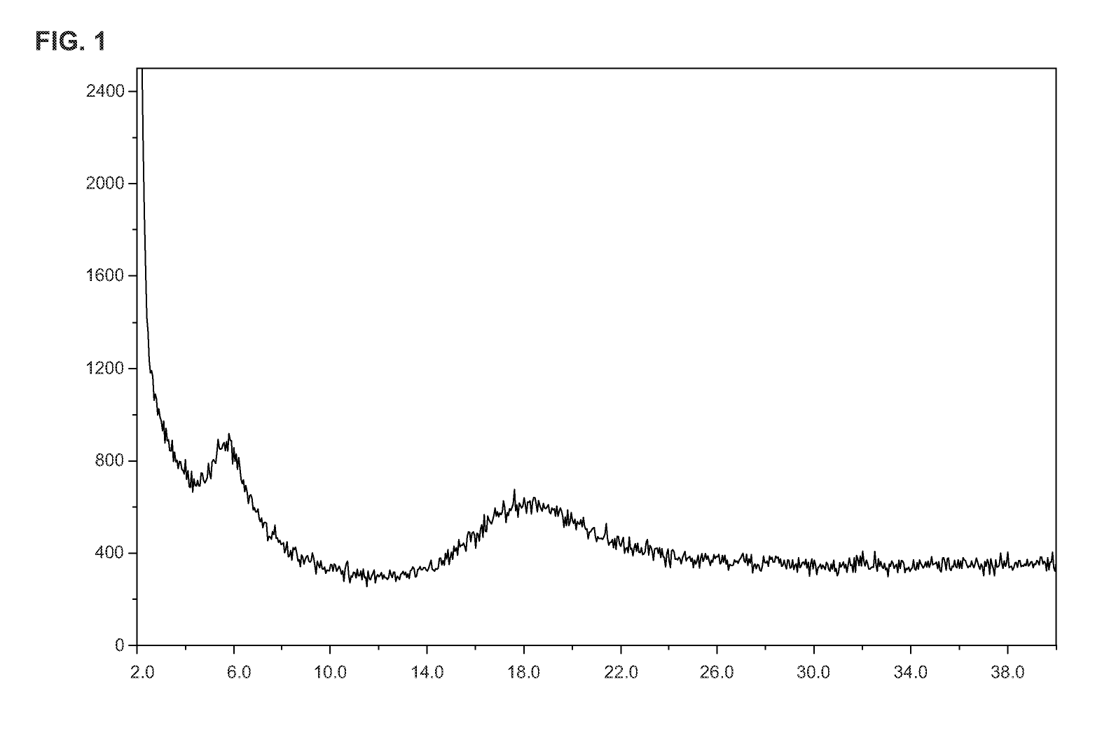

GBPE-Ca salt may be characterized by data selected from a group consisting of: ¹H NMR (CDCl₃+10% CD₃OD, 300 MHz): 6.8 (q, 5.4 Hz, 1H), 6.64 (brt, 5 Hz, 1H), 3.25 (brs, 2H), 2.52 (sept, 6.9 Hz, 1H), 2.17 (s, 2H), 1.46 (d, 4.2 Hz, 3H), 1.6-1.3 (m, 10H), 1.15 (d, 6.9 Hz, 6H); ¹³C NMR (CDCl₃+10% CD₃OD, 300 MHz): 187.67, 175.82, 155.30, 89.53, 47.05, 44.12, 36.82, 34.22, 34.15, 33.99, 26.10, 21.37, 18.61, 18.55; and Powder X-Ray Diffraction (“PXRD”) pattern as substantially depicted in FIG. 1.

The GBPE-Ca salt may be further characterized by MS (FAB−): m/z 328.1 (M−H).

Preferably, GBPE-Ca salt is characterized by ¹H NMR (CDCl₃+10% CD₃OD, 300 MHz): 6.8 (q, 5.4 Hz, 1H), 6.64 (brt, 5 Hz, 1H), 3.25 (brs, 2H), 2.52 (sept, 6.9 Hz, 1H), 2.17 (s, 2H), 1.46 (d, 4.2 Hz, 3H), 1.6-1.3 (m, 10H), 1.15 (d, 6.9 Hz, 6H); ¹³C NMR (CDCl₃+10% CD₃OD, 300 MHz): 187.67, 175.82, 155.30, 89.53, 47.05, 44.12, 36.82, 34.22, 34.15, 33.99, 26.10, 21.37, 18.61, 18.55; and MS (FAB−): m/z 328.1 (M−H).

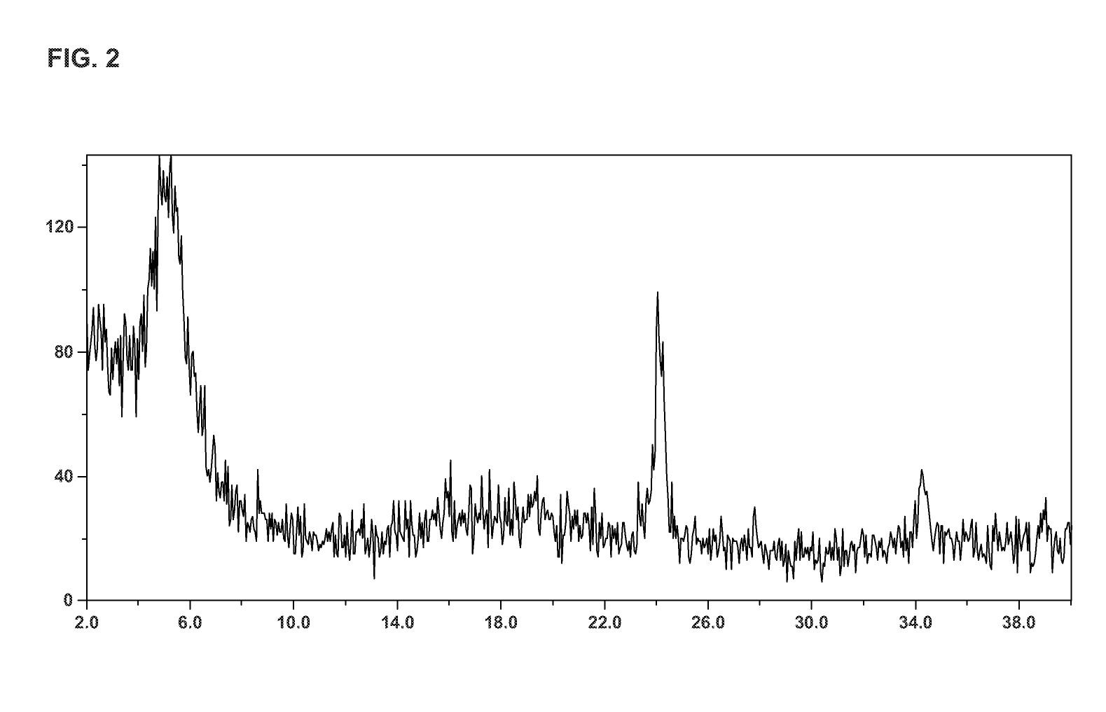

GBPE-Ba salt may be characterized by data selected from a group consisting of: ¹H NMR (CDCl₃, 400 MHz): 6.79 (brs, 1H), 6.18 (brs, 1H), 3.25 (brs, 2H), 2.52 (sept, 6.8 Hz, 1H), 2.17 (s, 2H), 1.46 (d, 4.8 Hz, 3H), 1.6-1.3 (m, 10H), 1.15 (d, 6.8 Hz, 6H); ¹³C NMR (CDCl₃, 300 MHz): 181.00, 175.26, 154.74, 89.06, 46.00, 36.56, 33.94, 33.65, 33.99, 26.10, 21.29, 19.24, 18.31; and PXRD pattern as substantially depicted in FIG. 2.

The GBPE-Ba salt may be further characterized by MS (FAB−): m/z 328.1 (M−H).

Preferably, GBPE-Ba salt is characterized by ¹H NMR (CDCl₃, 400 MHz): 6.79 (brs, 1H), 6.18 (brs, 1H), 3.25 (brs, 2H), 2.52 (sept, 6.8 Hz, 1H), 2.17 (s, 2H), 1.46 (d, 4.8 Hz, 3H), 1.6-1.3 (m, 10H), 1.15 (d, 6.8 Hz, 6H); ¹³C NMR (CDCl₃, 300 MHz): 181.00, 175.26, 154.74, 89.06, 46.00, 36.56, 33.94, 33.65, 33.99, 26.10, 21.29, 19.24, 18.31; and MS (FAB−): m/z 328.1 (M−H).

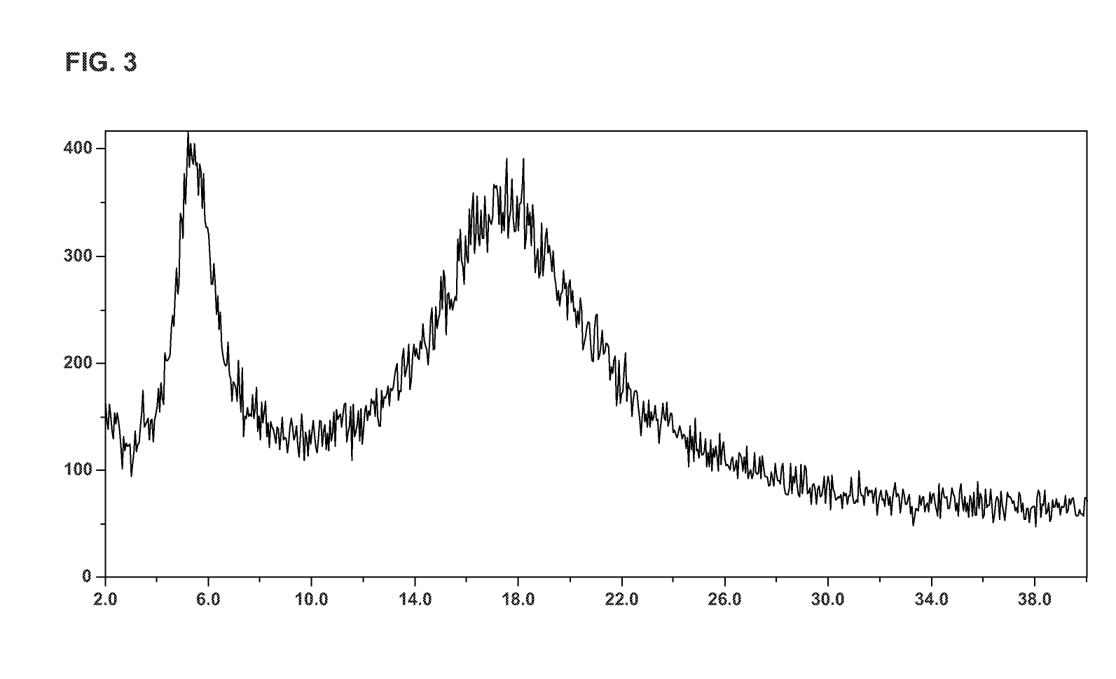

GBPE-Mg salt may be characterized by data selected from a group consisting of: ¹H NMR (CDCl₃, 400 MHz): 6.74 (brs, 1H), 6.05 (brs, 1H), 3.25 (m, 2H), 2.52 (sept, 6.4 Hz, 1H), 2.17 (s, 2H), 1.5-1.3 (m, 13H), 1.15 (d, 6.4 Hz, 6H); and ¹³C NMR (CDCl₃, 300 MHz): 174.84, 154.41, 89.17, 42.75, 37.023, 33.96, 33.60, 29.36, 25.79, 22.54, 21.25, 19.40, 18.41; and PXRD pattern as substantially depicted in FIG. 3.

Preferably, GBPE-Mg salt is characterized by ¹H NMR (CDCl₃, 400 MHz): 6.74 (brs, 1H), 6.05 (brs, 1H), 3.25 (m, 2H), 2.52 (sept, 6.4 Hz, 1H), 2.17 (s, 2H), 1.5-1.3 (m, 13H), 1.15 (d, 6.4 Hz, 6H); and ¹³C NMR (CDCl₃, 300 MHz): 174.84, 154.41, 89.17, 42.75, 37.023, 33.96, 33.60, 29.36, 25.79, 22.54, 21.25, 19.40, 18.41.

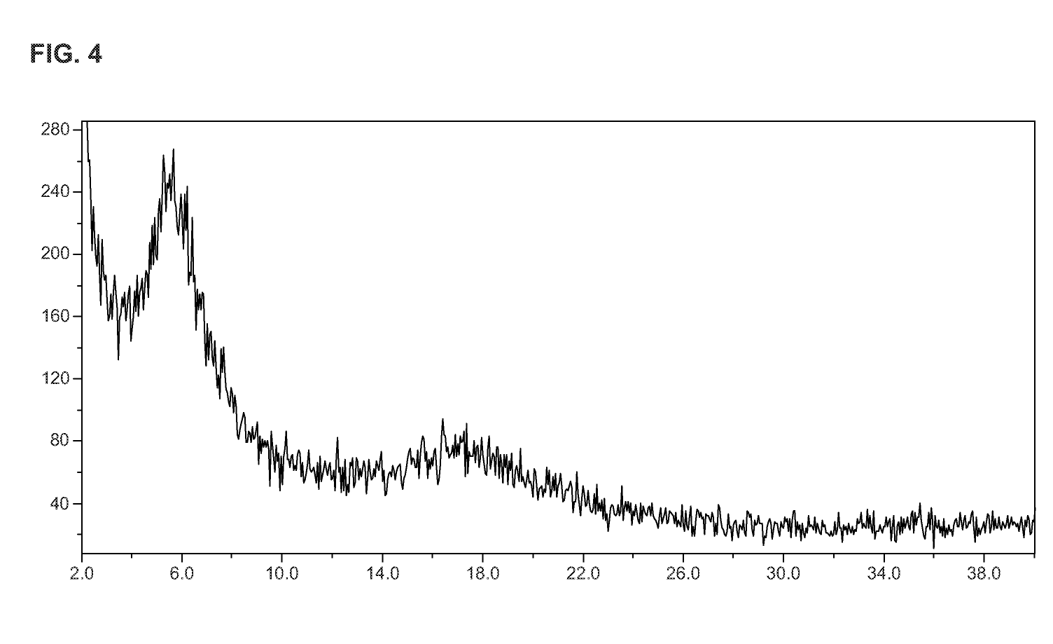

GBPE-Cu salt may be characterized by a PXRD pattern as substantially depicted in FIG. 4.

The GBPE-Cu salt may be further characterized by MS (FAB−): m/z 328.1 (M−H).

Preferably, GBPE-Cu salt is characterized by MS (FAB−): m/z 328.1 (M−H).

Additional GBPE salts may be any one of: GBPE-K, GBPE-Li, GBPE-Al or GBPE-Ag. The preferred GBPE salt is GBPE-Ca.

The present invention also encompasses solid GBPE salt selected from the group consisting of: GBPE-Ca, GBPE-Ba, GBPE-Mg and GBPE-Cu.

In addition, the present invention encompasses isolated GBPE salt selected from the group consisting of GBPE-Ca, GBPE-Ba, GBPE-Mg and GBPE-Cu.

The present invention also encompasses pure GBPE salt selected from the group consisting of GBPE-Ca, GBPE-Ba, GBPE-Mg and GBPE-Cu.

The present invention encompasses amorphous GBPE salt selected from the group consisting of GBPE-Ca, GBPE-Ba, GBPE-Mg and GBPE-Cu.

The present invention provides a process for preparing GBPE salt selected from the group consisting of GBPE-Ca, GBPE-Ba and GBPE-Mg comprising: dissolving GBPE in a water miscible organic solvent or mixtures of a water miscible organic solvent and water; adding a base selected from the group consisting of NaOH, KOH, K₂CO₃, KHCO₃, Na₂CO₃, NaHCO₃, LiOH, Li₂CO₃ and mixtures thereof; and adding CaCl₂ if the GBPE salt is GBPE-Ca, BaCl₂ if the GBPE salt is GBPE-Ba, or MgCl₂ if the GBPE salt is GBPE-Mg.

The process is exemplified for GBPE-Ca in the scheme below:

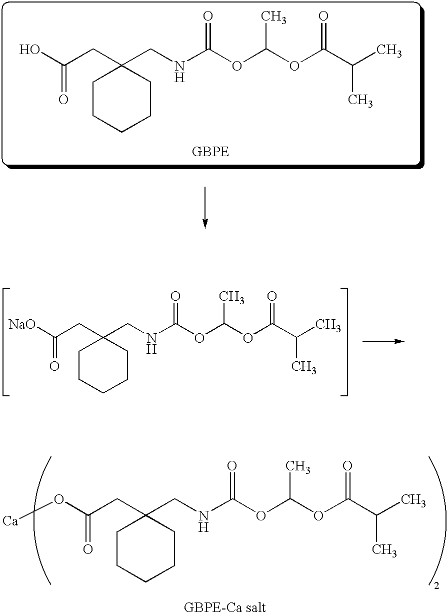

Preferably, the water miscible organic solvent is selected from the group consisting of: C₁-C₁₀ alcohol, dioxane, tetrahydrofuran (“THF”), acetone, dimethylformamide (“DMF”), dimethyl sulfoxide (“DMSO”), acetonitrile, and mixtures thereof. Preferably, the C₁-C₁₀ alcohol is ethanol or methanol. Optionally, methanol or ethanol is added with water.

Preferably, the salt is added as an aqueous solution.

Preferably, the process is done at a basic pH, which is typically of about 8 to about 14, preferably, between about 8 to about 10, more preferably, the pH is about 8.5.

Preferably, when the base is selected from a group consisting of: NaOH, KOH, NaHCO₃, KHCO₃ and LiOH, the ratio of GBPE and the base is of about 1:1. Preferably, when the base is selected from a group consisting of: Na₂CO₃, K₂CO₃, and Li₂CO₃, the ratio of GBPE and the base is of about 2:1.

Preferably, the base is NaOH.

Preferably, the base, e.g. NaOH, is added dropwise.

Optionally, following the addition of a base and prior to the salt addition, water is added.

Preferably, prior to the salt addition, and more preferably, after the water addition, the water miscible organic solvent is removed, preferably, by evaporation.

Preferably, the ratio of GBPE and the added salt is about 2:1.

GBPE of the above processes can be obtained by any method known in the art. Such a method is described for example in the U.S. Pat. No. 6,818,787, which is incorporated herein by reference.

The obtained GBPE salt may be further isolated. When the obtained product is GBPE Ca salt, the isolation may be done by filtration. Preferably, prior to the isolation a stirring step is performed. Preferably, the stirring is for about 4 hours to about 36 hours, more preferably, for about 6 hours to about 24 hours, most preferably, for about 8 to about 14 hours. The obtained product may be amorphous. After the isolation of the Ca salt of GBPE, mother liquor is obtained and may be further extracted.

The obtained salts GBPE Ca, GBPE Ba and GBPE Mg may be isolated by extraction. The extraction of the salts may be done with a water immiscible organic solvent having a boiling point of less than about 120° C. and thereafter evaporating. Preferably, the water immiscible organic solvent has a boiling point of above 40° C. Most preferably, the water immiscible organic solvent has a boiling point of about 40° C. to about 70° C. The water immiscible organic solvent may be selected from a group consisting of: chloroform, ethyl acetate, methyl tert-butyl ester (“MTBE”), CCl₄, toluene, CH₂Cl₂ and mixtures thereof, preferably, CH₂Cl₂.

In another embodiment, the present invention encompasses a process for preparing GBPE-Mg salt or GBPE-Cu salt comprising: dissolving GBPE in a water immiscible organic solvent; adding a base selected from the group consisting of NaOH, KOH, K₂CO₃, KHCO₃, Na₂CO₃, NaHCO₃, LiOH, Li₂CO₃, Cu(OAc)₂, Mg(OEt)₂ and mixtures thereof; and adding Cu(OAc)₂ if the GBPE salt is GBPE-Cu or Mg(OEt)₂ if the GBPE salt is GBPE-Mg.

The water immiscible organic solvent may be selected from a group consisting of: chloroform, ethyl acetate, methyl tert-butyl ester (“MTBE”), CCl₄, toluene, CH₂Cl₂ and mixtures thereof, preferably, CH₂Cl₂.

Preferably, when the base is selected from a group consisting of: NaOH, KOH, NaHCO₃, KHCO₃ and LiOH, the ratio of GBPE and the base is of about 1:1. Preferably, when the base is selected from a group consisting of: Na₂CO₃, K₂CO₃, Mg(OEt)₂, Cu(OAc)₂ and Li₂CO₃, the ratio of GBPE and the base is of about 2:1.

The rest of the parameters in this process are as described in the previous process, described above.

The present invention further provides a process for preparing GBPE-Mg salt or GBPE-Cu salt comprising: dissolving GBPE in a water immiscible organic solvent; and adding Cu(OAc)₂ if the GBPE salt is GBPE-Cu or Mg(OEt)₂ if the GBPE salt is GBPE-Mg. The water immiscible organic solvent is as described above.

Preferably, following the addition of Mg(OEt)₂ or Cu(OAc)₂, a stirring step is performed. Preferably, the stirring is at about room temperature to about 60° C. Preferably, the stirring is for about 12 hours to about 36 hours, more preferably, for about 24 hours.

The process may further comprise an isolation step. The isolation may be done by filtration. Optionally, the filtrate is evaporated to obtain GBPE Mg salt or GBPE Cu salt.

The GBPE salt selected from the group consisting of GBPE-Ca, GBPE-Ba, GBPE-Mg and GBPE-Cu may be used for seeding in a process for crystallizing GBPE.

The present invention encompasses a process for crystallizing GBPE by seeding it with a salt selected from the group consisting of GBPE-Ca, GBPE-Ba, GBPE-Mg and GBPE-Cu. Preferably, the process comprises combining the GBPE with a solvent to obtain a reaction mixture; and seeding the reaction mixture with a GBPE salt selected from the group consisting of GBPE-Ca, GBPE-Ba, GBPE-Mg and GBPE-Cu.

Optionally, the GBPE seeded is oil. When the GBPE seeded is an oil, the addition of a solvent is optional.

In one embodiment, the solvent may be selected from a group consisting of: a linear, branched or cyclic C₅-C₁₂ alkane, petroleum ether and mixtures thereof. The C₅-C₁₂ alkanes may be linear, branched or cyclic C₅-C₁₂ alkanes, such as pentane, hexane, heptane, octane, nonane, decane, undecane, dodecane, cyclohexane and methyl cyclohexane. Preferably, when the solvent is selected from the above list, not including undecane and cyclohexane, the process is done at a temperature of about −20° C. to about 35° C., preferably about −20° C. to about room temperature, more preferably about 10° C. to about room temperature. Preferably, when the solvent used is undecane, the process may be done at a temperature of about −15° C. to about room temperature. Preferably, when the solvent used is cyclohexane, the process may be done at a temperature of about 10° C. to about room temperature.

In another embodiment, the solvent may be selected from a group consisting of methyl ethyl ketone (“MEK”), chloroform, CH₂Cl₂, methylcyclopentyl ether, ethyl lactate and mixtures thereof, in a combination with a solvent selected from the group consisting of linear, branched or cyclic C₅-C₁₂ alkanes, petroleum ether and mixtures thereof. Optionally, the solvent is a mixture of heptane and CH₂Cl₂ or a mixture of heptane, hexane and EtOAc. The reaction mixture may be heated prior to the seeding step followed by a cooling step, especially when the solvent is either a mixture of heptane and CH₂Cl₂ or a mixture of heptane, hexane and EtOAc. Preferably, the heating is to a temperature of about 40° C. to about 75° C., preferably about 45° C. to about 65° C., more preferably about 50° C. to about 60° C., for example about 40° C. to about 65° C. Preferably, the cooling is to a temperature of about 35° C. to about 15° C., more preferably to about room temperature. Optionally, following the seeding step, the reaction mixture is further cooled to obtain a precipitate. Preferably, the cooling is to a temperature of about room temperature to about −40° C., preferably room temperature to −30° C., more preferably about room temperature to about −20° C., yet more preferably about 10° C. to about −20° C., yet more preferably about 0° C. to about −20° C. For example, about 15° C. to about −30° or about 0° C. to about −25° C., preferably to about −20° C. Optionally, the precipitate is further slurried in a C₅ to C₁₂ hydrocarbon solvent, preferably, in a C₅ to C₈ hydrocarbon solvent. Preferably, the hydrocarbon solvent is heptane or hexane, more preferably, n-heptane. The obtained product may be isolated, preferably by filtration.

The product obtained from the above processes is solid and may be further used for seeding in crystallization processes of GBPE.

The crystalline GBPE obtained according to the above process may be pure.

The present invention further encompasses 1) a pharmaceutical composition comprising any one, or combination, of GBPE salts described above and at least one pharmaceutically acceptable excipient and 2) the use of any one, or combination, of the above-described GBPE salts and/or amorphous form, in the manufacture of a pharmaceutical composition, wherein the pharmaceutical composition can be useful for the treatment of cerebral diseases such as epilepsy, allodynia, or hyperalgesia.

The pharmaceutical composition of the present invention can be in a solid or a non-solid form. If the pharmaceutical composition is in a non-solid form, any one, or combination, of the GBPE salts within the composition, are retained as solid(s) in the non-solid pharmaceutical composition, e.g., as a suspension, foam, ointment and etc.

The pharmaceutical composition can be prepared by a process comprising combining any one, or combination, of the above-described GBPE salts with at least one pharmaceutically acceptable excipient. The GBPE salts can be obtained by any of the processes of the present invention as described above.

The pharmaceutical composition can be used to make appropriate dosage forms such as tablets, powders, capsules, suppositories, sachets, troches and losenges.

Any one, or combination, of the above-described GBPE salts of the present invention, particularly in a pharmaceutical composition and dosage form, can be used to treat cerebral diseases in a mammal such as a human, comprising administering a treatment effective amount of GBPE salts in the mammal. The treatment effective amount or proper dosage to be used can be determined by one of ordinary skill in the art, which can depend on the method of administration, the bioavailability, the age, sex, symptoms and health condition of the patient, and the severity of the disease to be treated, etc.

Having thus described the invention with reference to particular preferred embodiments and illustrative examples, those in the art can appreciate modifications to the invention as described and illustrated that do not depart from the spirit and scope of the invention as disclosed in the specification. The Examples are set forth to aid in understanding the invention but are not intended to, and should not be construed to, limit its scope in any way. Absent statement to the contrary, any combination of the specific embodiments described above are consistent with and encompassed by the present invention.

### Instruments

¹H-NMR and ¹³C-NMR
¹H-NMR and ¹³C-NMR spectra were obtained on Bruker AM-300 and DMX-600 spectrometers.

Mass Spectrometry

Mass spectrometry results obtained from a Finnigan 4000 spectrometer.

Powder X-Ray Diffraction
1) Powder X-ray diffraction data was obtained by using an ARL instrument, model X'TRA-019, Peltier detector, round standard aluminum sample holder with round zero background quartz plate. The cathode is CuKa radiation, λ=1.5418 Å.
Sample: Spin/Oscillate mode
Range: 2-40 degrees two-theta
Scan mode: Continuous scan
Step size: 0.05 deg
Scan rate: 3 deg./min
2) Additional powder X-ray diffraction data was obtained from Bruker X-Ray powder diffractometer model D8 advance equipped with lynxEye. λ=1.5418 Å.
The accuracy of peak positions is defined as ±0.2 degrees due to experimental differences such as instrumentations and sample preparations.

Thermogravimetric Analysis (“TGA”)
TGA data obtained from TGA/DSC 1 of Mettler-Toledo or TA Instruments TGA 2959
Heating range: 25-200 C; heating rate: 10° C./min,
Nitrogen flow 40 ml/min
Mass weight about 10 mg.

### EXAMPLES

### Example 1

### Preparation of GBPE-Ca salt

1-{[(α-Isobutanoyloxyethoxy)carbonyl]-aminomethyl}-1-cyclohexane acetic acid [GBPE] (1.3 g, 3.95 mmol) was dissolved in solution of EtOH/water=2:1 (12 mL) followed by dropwise addition of 1M NaOH (3 mL) until the pH reached approximately 8.5. The obtained solution was evaporated and water (10 mL) was added. Aqueous CaCl₂ (20 mL, 3 mmol) was added to give milky solution that was stirred over night. The off-white precipitate was formed and collected by suction filtration. This solid was analyzed by XRD and found to be amorphous Form of GBPE-Ca salt.

The mother liquor was extracted with CH₂Cl₂ and evaporated to give bright yellow powder which was analyzed by XRD and found to be Amorphous GBPE-Ca salt. The total yield of the product obtained from the two phases was 63%.

### Example 2

### Preparation of GBPE-Ba salt

1-{[(α-Isobutanoyloxyethoxy)carbonyl]-aminomethyl}-1-cyclohexane acetic acid [GBPE](0.5 g, 1.5 mmol) was dissolved in solution of EtOH/water=2:1 (6 mL) followed by dropwise addition of 1M NaOH (1.5 mL) until the pH reached approximately 8.5 and then evaporated. Water (10 mL) and aqueous BaCl₂ (20 mL, 3 mmol) were added and the resulting solution was stirred at room temperature for over night. The reaction was washed with CH₂Cl₂ (10 mL) and the organic phase was evaporated to give yellow solid (0.2 g), which was analyzed by XRD and found to be amorphous Form of GBPE-Ba salt.

### Example 3

### Preparation of GBPE-Mg Salt

1-{[(α-Isobutanoyloxyethoxy)carbonyl]-aminomethyl}-1-cyclohexane acetic acid [GBPE] (0.5 g, 1.5 mmol) was dissolved in solution of EtOH/water=2:1 (6 mL) followed by dropwise addition of 1M NaOH (1.5 mL) until the pH reached approximately 8.5 and then evaporated. Water (10 mL) and aqueous MgCl₂ (20 mL, 3 mmol) were added and the resulting solution was stirred at room temperature for over night. The reaction was washed with CH₂Cl₂ (10 mL) and the organic phase was evaporated to give yellow solid (0.25 g), which was analyzed by XRD and found to be amorphous Form of GBPE-Mg salt.

### Example 4

### Preparation of GBPE Mg Salt

GBPE (0.5 g, 1.52 mmol) was dissolved in toluene (5 ml) followed by addition of Mg(OEt)2 (0.5 g). The obtained mixture was stirred at room temperature for 24 h. The reaction was stopped, filtered and the filtrate was evaporated to give GBPE Mg salt in quantitative yield.

### Example 5

### Preparation of GBPE-Cu Salt

1-{[(α-Isobutanoyloxyethoxy)carbonyl]-aminomethyl}-1-cyclohexane acetic acid [GBPE] (0.5 g, 1.5 mmol) was dissolved in CH₂Cl₂ (10 mL) followed by addition of aqueous NaOH (1.5 mmol, 10 mL). The resulting mixture was stirred vigorously and the aqueous Cu(OAc)₂ was added. The obtained turbid solution was stirred at 25° C. for over night. The layers were separated and the organic phase was evaporated to give turquoise solid (0.35 g) which was analyzed by XRD and found to be amorphous Form of GBPE-Cu salt.

### Example 6

### Solubility Test

Solubility was determined by placing 100 mg of sample material in a 50 ml glass beaker, adding 10 μL portions of distilled water and agitating until dissolved. The amount of added water was calculated to obtain the solubility of each salt, and the results are summarized in Table 1 below.

TABLE 1
Solubility of GBPE salts and temperature at which thermal
decomposition starts.

| | | Temperature at which |
|---|---|---|
| | Solubility (g/ml | decomposition starts/ |
| Tested material | water) | ° C. |
| GBPE-Ca salt | 0.016 | 70 |
| | sparingly soluble | |
| GBPE-Mg salt | 0.020 | 70 |
| | sparingly soluble | |
| GBPE-Ba salt | 0.125 | 60 |
| | freely soluble | |
| GBPE-Cu salt | 0.0125 | / |
| | sparingly soluble | |
| GBPE amorphous | insoluble | 30 |

### Example 7

### Crystallization of GBPE

### Method 1:

1-{[(α-Isobutanoyloxyethoxy)carbonyl]-aminomethyl}-1-cyclohexane acetic acid [GBPE] (0.7 g, 2.12 mmol) as a yellow oil was heated to a temperature of about 50° C.-60° C. in a mixture of Heptane/CH₂Cl₂=5:1 for 5-30 min and then cooled to room temperature to give oily residue. This residue was seeded at room temperature with GBPE-Ca salt. The mixture was cooled at −20° C. for 12 h to 48 h. The obtained solid material was slurried in ice-cold Heptane and collected by suction filtration to give solid GBPE (0.45 g, 64% yield).

### Method 2:

1-{[(α-Isobutanoyloxyethoxy)carbonyl]-aminomethyl}-1-cyclohexane acetic acid [GBPE] (0.65 g, 1.97 mmol) as a yellow oil was heated to 60° C. in a mixture of Heptane/Hexane/EtOAc=5:5:1 for 5-30 min and then cooled to room temperature to give oily residue. This residue was cooled at −78° C. followed by seeding with GBPE-Ca salt. The flask was then brought to −20° C. for 12 h to 48 h. The obtained solid material was slurried in ice-cold Heptane and collected by suction filtration to give solid GBPE (0.32 g, 50% yield).

### Method 3:

1-{[(α-Isobutanoyloxyethoxy)carbonyl]-aminomethyl}-1-cyclohexane acetic acid [GBPE] (2.8 g, 8.5 mmol) as a yellow oil was heated to 60° C. in a mixture of Heptane/CH₂Cl₂=5:1 for 5-30 min and then cooled to room temperature to give oily residue. This residue was seeded at room temperature_with pure crystalline GBPE [obtained by experimental procedure described in Method 1 and Method 2]. The flask was cooled at −20° C. for 12 h to 48 h. The obtained solid material was slurried in ice-cold Heptane and collected by suction filtration to give solid GBPE (1.5 g, 54% yield).

### Method 4:

1-{[(α-Isobutanoyloxyethoxy)carbonyl]-aminomethyl}-1-cyclohexane acetic acid [GBPE] (1.8 g, 5.4 mmol) as a yellow oil was seeded with pure crystalline GBPE [obtained by experimental procedure described in Method 1 and Method 2] followed by addition of Hexane. The obtained residue was evaporated to dryness to give sticky solid which was crushed and slurried in Hexane. The obtained solid material was collected by suction filtration to give solid GBPE (1.3 g, 72% yield).

### Method 5:

1-{[(α-Isobutanoyloxyethoxy)carbonyl]-aminomethyl}-1-cyclohexane acetic acid [GBPE] (8.7 g, 26.4 mmol) as a yellow oil was cooled at −20° C. for 12 h to 48 h to form crystalline material. The obtained solid was slurried in Hexane and then the crystalline material was collected by suction filtration to give solid GBPE (8.3 g, 95.4% yield).

Methods 6A to 38C—General procedure 1

1-{[(α-Isobutanoyloxyethoxy)carbonyl]-aminomethyl}-1-cyclohexane acetic acid [GBPE] as a yellow oil is mixed in a solvent (see Table 1) at a specific temperature (see Table 1) followed by either seeding with GBPE-Ca salt, seeding with pure GBPE or no seeding at all (see Table 1) for 12 h. The obtained solid is collected by suction filtration.

TABLE 1

| | | | Seeding agent | | |
|---|---|---|---|---|---|
| | | | | GBPE- | |
| | Solvent | Temperature | No seeding | Ca salt | GBPE |
| METHOD | Pentane | RT | 6A | 17A | 28A |
| NUMBER | | 0° C. | 6B | 17B | 28B |
| | | −20° C. | 6C | 17C | 28C |
| | Hexane | RT | 7A | 18A | 29A |
| | | 0° C. | 7B | 18B | 29B |
| | | −20° C. | 7C | 18C | 29C |
| | Heptane | RT | 8A | 19A | 30A |
| | | 0° C. | 8B | 19B | 30B |
| | | −20° C. | 8C | 19C | 30C |
| | Octane | RT | 9A | 20A | 31A |
| | | 0° C. | 9B | 20B | 31B |
| | | −20° C. | 9C | 20C | 31C |
| | Nonane | RT | 10A | 21A | 32A |
| | | 0° C. | 10B | 21B | 32B |
| | | −20° C. | 10C | 21C | 32C |
| | Decane | RT | 11A | 22A | 33A |
| | | 0° C. | 11B | 22B | 33B |
| | | −20° C. | 11C | 22C | 33C |
| | Undecane | RT | 12A | 23A | 34A |
| | | 0° C. | 12B | 23B | 34B |
| | | −15° C. | 12C | 23C | 34C |
| | Dodecane | RT | 13A | 24A | 35A |
| | | 0° C. | 13B | 24B | 35B |
| | Cyclohexane | RT | 14A | 25A | 36A |
| | | 10° C. | 14B | 25B | 36B |
| Method | Methyl | RT | 15A | 26A | 37A |
| number | cyclohexane | 0° C. | 15B | 26B | 37B |
| | | −20° C. | 15C | 26C | 37C |
| | Petroleum | RT | 16A | 27A | 38A |
| | Ether | 0° C. | 16B | 27B | 38B |
| | 40-60 | −20° C. | 16C | 27C | 38C |

### Methods 39B to 68C—General Procedure 2

1-{[(α-Isobutanoyloxyethoxy)carbonyl]-aminomethyl}-1-cyclohexane acetic acid [GBPE] as a yellow oil is mixed in a solvent (see Table 2) at a specific temperature (see Table 2) followed by either seeding with GBPE-Ca salt, seeding with pure GBPE or no seeding at all (see Table 2) for 12 h. The obtained solid is collected by suction filtration.

TABLE 2

| | | | Seeding agent | | |
|---|---|---|---|---|---|
| | Solvent | Temperature | No seeding | GBPE-Ca | GBPE |
| Method | MEK/hexane | RT | — | 49A | 59A |
| number | | 0° C. | 39B | 49B | 59B |
| | | −20° C. | 39C | 49C | 59C |
| | MEK/heptane | RT | 40A | 50A | 60A |
| | | 0° C. | 40B | 50B | 60B |
| | | −20° C. | 40C | 50C | 60C |
| | CHCl₃/Hexane | RT | 41A | 51A | 61A |
| | | 0° C. | 41B | 51B | 61B |
| | | −20° C. | 41C | 51C | 61C |
| | | | Seeding agent | | |
| | | | | GBPE-Ca | |
| | Solvent | Temperature | No seeding | salt | GBPE |
| METHOD | CHCl₃/Heptane | RT | 42A | 52A | 62A |
| NUMBER | | 0° C. | 42B | 52B | 62B |
| | | −20° C. | 42C | 52C | 62C |
| | CH₂Cl₂/hexane | RT | — | 53A | 63A |
| | | 0° C. | — | 53B | 63B |
| | | −20° C. | — | 53C | 63C |
| | CH₂Cl₂/ | 0° C. | 43B | — | — |
| | Cyclohexane | −20° C. | 43C | — | — |
| | CH₂Cl₂/heptane | RT | 44A | 54A | 64A |
| | | 0° C. | 44B | 54B | 64B |
| | | −20° C. | 44C | 54C | 64C |
| | Methylcyclopentyl | RT | — | 55A | 65A |
| | Ether/pentane | 0° C. | 45B | 55B | 65B |
| | | −20° C. | 45C | 55C | 65C |
| | Methylcyclopentyl | RT | 46A | 56A | 66A |
| | Ether/pentane | 0° C. | 46B | 56B | 66B |
| | | −20° C. | 46C | 56C | 66C |
| | Ethyl lactate/ | RT | 47A | 57A | 67A |
| | hexane | 0° C. | 47B | 57B | 67B |
| | | −20° C. | 47C | 57C | 67C |
| | Ethyl lactate/ | RT | 48A | 58A | 68A |
| | heptane | 0° C. | 48B | 58B | 68B |
| | | −20° C. | 48C | 58C | 68C |

### Method 39A:

1-{[(α-Isobutanoyloxyethoxy)carbonyl]-aminomethyl}-1-cyclohexane acetic acid [GBPE] (30 mg, 0.09 mmol) as a yellow oil was slurried for 24 h at room temperature in a combination of MEK/hexane=1:10. The obtained solid was collected by centrifugal filtration to give solid GBPE (24 mg, 80% yield).

### Method 41A*

1-{[(α-Isobutanoyloxyethoxy)carbonyl]-aminomethyl}-1-cyclohexane acetic acid [GBPE] (30 mg, 0.09 mmol) as a yellow oil was slurried at room temperature in a combination of Chloroform/petroleum ether (40-60° C.)=1:10. The obtained solid was collected by centrifugal filtration to give solid GBPE (24 mg, 80% yield).

### Method 43A:

1-{[(α-Isobutanoyloxyethoxy)carbonyl]-aminomethyl}-1-cyclohexane acetic acid [GBPE] (0.2 g, 0.6 mmol) as a yellow oil was slurried at room temperature in a combination of CH₂Cl₂/cyclohexane=1:10. The obtained solid was collected by suction filtration to give solid GBPE (0.1 g, 50% yield).

### Method 45A:

1-{[(α-Isobutanoyloxyethoxy)carbonyl]-aminomethyl}-1-cyclohexane acetic acid (30 mg, 0.09 mmol) as a yellow oil was slurried at room temperature in a combination of Methylcyclopentyl ether/pentane=1:10. The obtained solid was collected by centrifugal filtration to give solid GBPE (6 mg, 20% yield).

1. A GBPE salt selected from the group consisting of: GBPE-Ca salt, GBPE-Ba salt, GBPE-Mg salt and GBPE-Cu salt.

2. The GBPE salt of claim 1, where the salt is GBPE-Ca salt.

3. The GBPE salt of claim 1, where the salt is GBPE-Ba salt.

4. The GBPE salt of claim 1, where the salt is GBPE-Mg salt.

5. The GBPE salt of claim 1, where the salt is GBPE-Cu salt.

6. The GBPE salt of claim 1, wherein the salt is in a solid form.

7. The GBPE salt of claim 1, wherein the salt is in an isolated form.

8. The GBPE salt of claim 1, wherein the salt is in its pure form.

9. The GBPE salt of claim 1, wherein the salt is in its amorphous form.

10. A process for preparing GBPE-Mg salt or GBPE-Cu salt comprising: dissolving GBPE in a water immiscible organic solvent; adding a base selected from the group consisting of NaOH, KOH, K₂CO₃, KHCO₃, Na₂CO₃, NaHCO₃, LiOH, Li₂CO₃, Cu(OAc)₂, Mg(OEt)₂ and mixtures thereof; and adding Cu(OAc)₂ if the GBPE salt is GBPE-Cu or Mg(OEt)₂ if the GBPE salt is GBPE-Mg.

11. A process for preparing GBPE-Mg salt or GBPE-Cu salt comprising: dissolving GBPE in a water immiscible organic solvent; and adding Cu(OAc)₂ if the GBPE salt is GBPE-Cu or Mg(OEt)₂ if the GBPE salt is GBPE-Mg.

12. The process of claim 10, wherein the water immiscible organic solvent is selected from a group consisting of: chloroform, ethyl acetate, methyl tert-butyl ester (“MTBE”), CCl₄, toluene, CH₂Cl₂ and mixtures thereof.

13. A process for preparing GBPE salt selected from the group consisting of GBPE-Ca, GBPE-Ba and GBPE-Mg comprising: dissolving GBPE in a water miscible organic solvent or mixtures of a water miscible organic solvent and water; adding a base selected from the group consisting of NaOH, KOH, K₂CO₃, KHCO₃, Na₂CO₃, NaHCO₃, LiOH, Li₂CO₃ and mixtures thereof; and adding CaCl₂ if the GBPE salt is GBPE-Ca, BaCl₂ if the GBPE salt is GBPE-Ba, or MgCl₂ if the GBPE salt is GBPE-Mg.

14. The process of claim 13, wherein the water miscible organic solvent is selected from the group consisting of: C₁-C₁₀ alcohols, dioxane, tetrahydrofuran (“THF”), acetone, dimethylformamide (“DMF”), dimethyl sulfoxide (“DMSO”), acetonitrile, and mixtures thereof.

15. The process of claim 14, wherein the water miscible organic solvent is ethanol, methanol, or a mixture of ethanol and/or methanol with water.

16. The process of claim 10, wherein the process is done at a pH of about 8 to about 14.

17. The process of claim 10, wherein the base is NaOH.

18. The process of claim 10, wherein prior to the salt addition, water is added.

19. The process of claim 10, wherein prior to the salt addition, the solvent is removed.

20. A process for crystallizing GBPE comprising seeding a GBPE-containing reaction mixture with a salt selected from the group consisting of GBPE-Ca, GBPE-Ba, GBPE-Mg and GBPE-Cu.

21. The process of claim 20, wherein the process comprises combining the GBPE with a solvent to obtain a reaction mixture; and seeding the reaction mixture with a GBPE salt selected from the group consisting of GBPE-Ca, GBPE-Ba, GBPE-Mg and GBPE-Cu.

22. The process of claim 21, wherein the solvent is selected from a group consisting of: linear, branched or cyclic C₅-C₁₂ alkane, petroleum ether and mixtures thereof.

23. The process of claim 21, wherein the solvent is selected from a group consisting of methyl ethyl ketone (“MEK”), chloroform, CH₂Cl₂, methylcyclopentyl, ethyl lactate and mixtures thereof, in a combination with a solvent selected from the group consisting of linear, branched or cyclic C₅-C₁₂ alkanes, petroleum ether and mixtures thereof.

24. The process of claim 23, wherein the solvent is a mixture of heptane and CH₂Cl₂ or a mixture of heptane, hexane and EtOAc.

25. The process of claim 20, wherein the reaction mixture is heated prior to the seeding step and wherein the reaction mixture is cooled after the heating step.

26. The process of claim 25, wherein the reaction mixture is heated to a temperature of about 40° C. to about 75° C.

27. The process of claim 25, wherein the cooling is to a temperature of about 35° C. to about 15° C.

28. The process of claim 23, further comprising a cooling step sufficient to obtain a precipitate.

29. The process of claim 28, wherein the cooling is to a temperature of about room temperature to about −40° C.

30. The process of claim 28, wherein the precipitate is slurried in a C₅ to C₁₂ hydrocarbon solvent.

31. A pharmaceutical composition comprising GBPE salt selected from a group consisting of: GBPE-Ca salt, GBPE-Ba salt, GBPE-Mg salt and GBPE-Cu salt.

32. (canceled)
